(19) United States
(12) Patent Application Publication
(10) Pub. No.: US 2026/0335164 A1
(43) Pub. Date: Oct. 29, 2026

(54) Linear Exoskeleton for Reducing Knee Contact Force

(71) Applicant: The Board of Trustees of the Leland Stanford Junior University, Stanford, CA (US)

(72) Inventors: Patrick Slade, Somerville, MA (US); Scott l. Delp, Stanford, CA (US); Nicholas Miguel Robles, San MAteo, CA (US)

(21) Appl. No.: 19/118,914

(22) PCT Filed: Oct. 12, 2023
(86) PCT No.: PCT/US2023/035034

§ 371 Date: Apr. 7, 2025

Related U.S. Application Data
(60) Provisional application No. 63/415,585, filed on Oct. 12, 2022.

(51) Int. Cl. A61H 1/02; A61H 3/00

(52) U.S. Cl. CPC A61H 1/0237; A61H 2003/007; A61H 2201/1642; A61H 2201/1664; A61H 2201/5061; A61H 2201/5066

(57) ABSTRACT

A wearable robotic device, called linear exoskeleton, is provided. The linear exoskeleton applies active assistance to reduce the knee contact forces during walking. The objective of Pulley assembly the exoskeleton is to reduce knee contact force by using a motor to apply forces between a strap on the thigh and the ground. The force Force sensor applied is controllable. The exoskeleton itself has a linear actuator type design, with a carriage that slides along a carbon pole.

### FIELD OF THE INVENTION

This invention relates to exoskeletons. In particular, the invention relates to linear exoskeletons.

### BACKGROUND OF THE INVENTION

Knee osteoarthritis is a condition where cartilage loss and bone spur development in the knee cause people significant pain. Knee osteoarthritis is the most common cause of adult musculoskeletal pain. This pain occurs when the knee is put under loads, such as walking and running, when there has been loss of articular cartilage in the knee and the development of bone spurs due to age and continual use of the leg. If the cartilage structure becomes damaged, repetitive loads during walking can further degrade the cartilage, causing a negative spiral in cartilage health (for reference, see FIG. 1 in priority document).

Approximately 40% of adults over the age of 55 have knee osteoarthritis. Reducing the forces in the knee when moving can significantly reduce the pain of the person. Thus, reducing knee contact force can slow the progression of osteoarthritis as well as improve people's quality of life when they have osteoarthritis. Existing solutions on the market are devices like medial unloader braces, which provide small reductions in knee contact force (<7%). The present invention provides new technology to assist in reducing knee contact force.

### SUMMARY OF THE INVENTION

The present invention provides a linear exoskeleton for reducing knee contact force. The linear exoskeleton has a pole with a distal end and a proximal end. In one example, the pole is a linear carbon pole. In one example, the pole is positioned at a lateral side of the person. The linear exoskeleton further has a shoe attachment at the distal end of the pole and capable to be attached to a shoe. In one example, the attachment to the shoe is adjustable. A carriage is positioned near the proximal end of the pole, such that the pole is capable of moving relative to the carriage in up and down position. The linear exoskeleton further has a thigh strap near the proximal end of the pole, such that the thigh strap is capable to be affixed the pole to a person thigh and capable of positioning the pole adjacent to a person's leg. A mechanical assembly is positioned at the proximal end of the pole, such that the mechanical assembly is mechanically connected to the carriage. A motor then controls the relative distance between the assembly and the carriage, therewith capable of controlling an assistive force to the person and therewith capable of reducing a knee contact force of the person while the person is executing a leg movement and in contact with a ground surface.

In one embodiment, the linear exoskeleton has a sensor for sensing contact with the ground surface. Examples of sensors are an Adafruit LSM9DS1 Accelerometer+Gyro+Magnetometer 9-DOF.

In another embodiments, the linear exoskeleton has a sensor for sensing the assistive force by the pole. Example of a sensor is a Futek Model LCM200 Ultra-Light Miniature Universal Threaded Load Cell.

In yet another embodiment, the linear exoskeleton has a controller for controlling the motor according to a desired force contact profile of the leg movement and receiving feedback from one or more sensors regarding the assistive force and ground contact. The controller further has a first calibration module for static calibration of the controller when no leg movement or ground contact is experienced. The controller further has a second calibration module for dynamic calibration of the controller during execution of the leg movement. The controller further has an input of body weight of the person and an algorithm to scale the desired force contact profile. The controller further has a scaling factor and an algorithm to scale the desired force contact profile. The controller further has input from one or more sensors and an algorithm to determine a type of leg movement, a desired force contact profile and a scaling factor to scale the desired force profile.

### BRIEF DESCRIPTION OF THE DRAWINGS

FIG. 1 shows according to an exemplary embodiment of the invention a linear exoskeleton worn by a person. The linear exoskeleton applies a vertical force to offload the knee by lifting the person with a strap on their thigh and applying the reaction force directly into the ground. The exoskeleton has a carriage attached to a strap on the thigh. The carriage rolls along a carbon tube that runs from the hip down to the foot, where it attaches to the shoe. The carbon tube transmits force to the carriage with a Bowden cable that runs over a pulley attached to the top of the carbon tube. A motor (not shown in the figure) applies a force to the cable.

FIG. 2 shows according to an exemplary embodiment a side view of the linear exoskeleton assembly.

FIG. 3 shows according to an exemplary embodiment the linear exoskeleton applying force to the leg by using a motor to wind a cord. This cord travels through a sheath, or Bowden cable, to transmit forces directly to the linear exoskeleton. The cable sheath terminates on a carriage, which slides along the pole of the exoskeleton. The cable runs over a pulley mounted on top of the exoskeleton pole, and terminates on the other side of the carriage with a load cell sensor that measures the force the device is applying to the exoskeleton. Thus, as force is applied by the motor, it attempts to wind in the cable on the exoskeleton, raising the carriage upwards toward the top of the tube. The strap attached to the carriage transmits force to the person's thigh.

FIG. 4 shows according to an exemplary embodiment a closeup on the foot assembly of the linear exoskeleton. The force through the thigh strap is transferred down the tube directly to the ground, completely offloading the leg. The inertial measurement unit (IMU) is attached at the base of the pole to detect when the foot is on the ground and where the foot is within the gait cycle. The foot support places force outside the foot print, improving mediolateral stability of the person by widening their support beyond the foot outline.

FIG. 5 shows according to an exemplary embodiment ground reaction forces between the foot and ground during walking on level-ground, incline, and decline. The linear exoskeleton force profiles follow a similar shape, scaled to a specified level of assistance.

FIG. 6 shows according to an exemplary embodiment a force profile applied by the linear exoskeleton with each step. The force profile is proportional to the force the person applies to the ground.

FIG. 7 shows according to an exemplary embodiment the linear exoskeleton controller withe linear exoskeleton sensors (force measurement sensors and inertial measurement unit), the motor that applies the force, and the software controlling the exoskeleton.

FIG. 8 shows according to an exemplary embodiment assistance through the linear exoskeleton reducing the knee contact force proportionally to the amount of assistance applied. R, G, B, M indicators indicate the bold lines.

### DETAILED DESCRIPTION

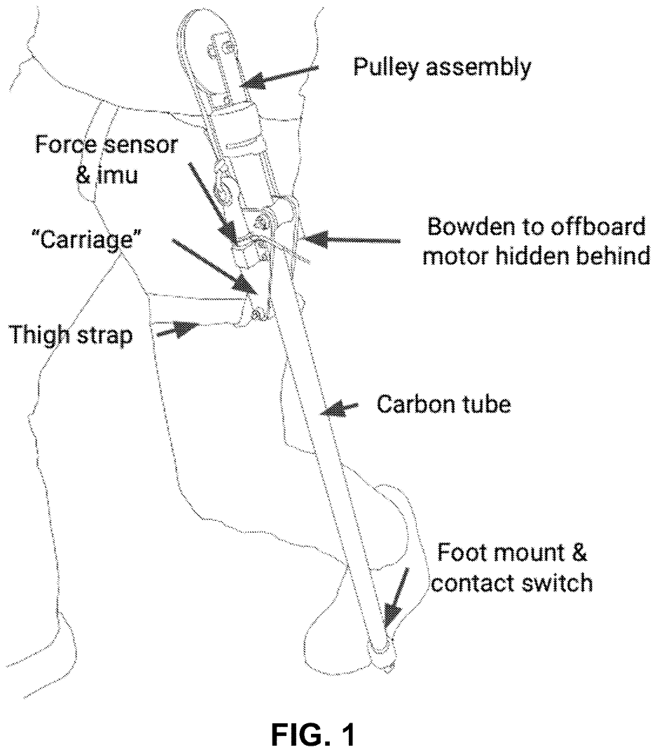

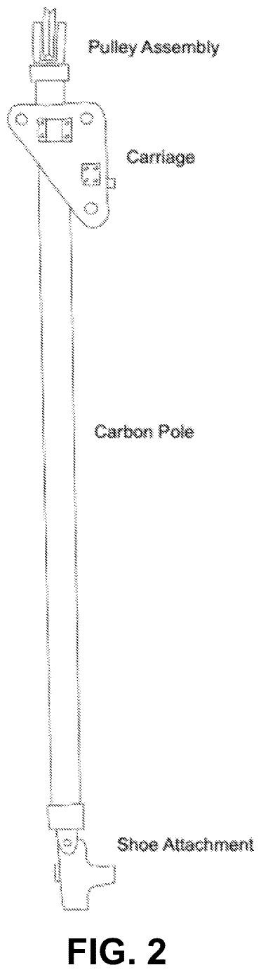

The present invention is a wearable robotic device, called linear exoskeleton. The linear exoskeleton applies active assistance to reduce the knee contact forces during walking. FIG. 1 shows an embodiment of the linear exoskeleton while attached by a person. Individual components of the linear exoskeleton are shown as well for this embodiment. The objective of the exoskeleton is to reduce knee contact force by using a motor to apply forces between a strap on the thigh and the ground. The force applied is controllable. The exoskeleton itself has a linear actuator type design, with a carriage that slides along a carbon pole (FIG. 2).

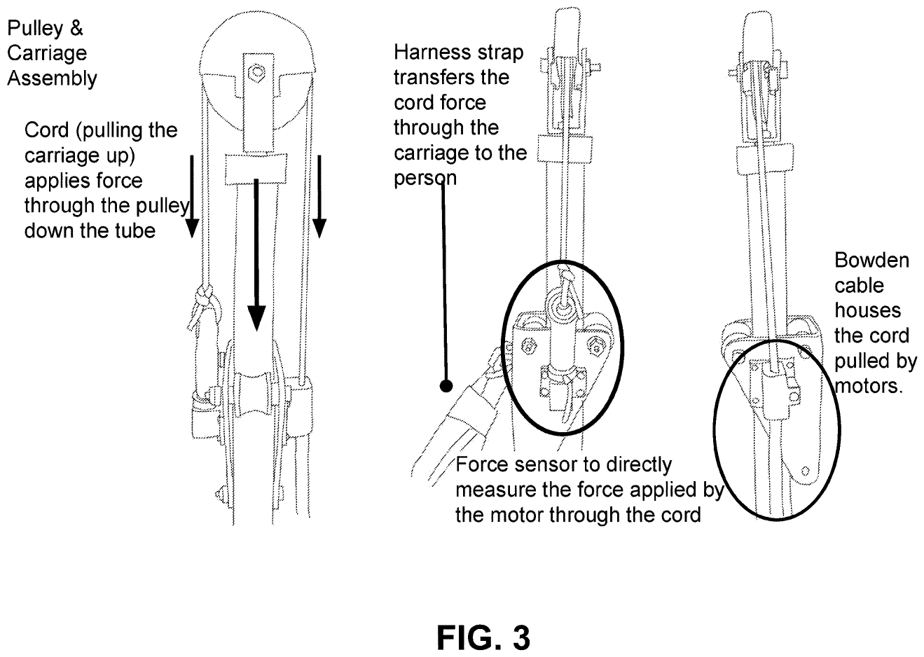

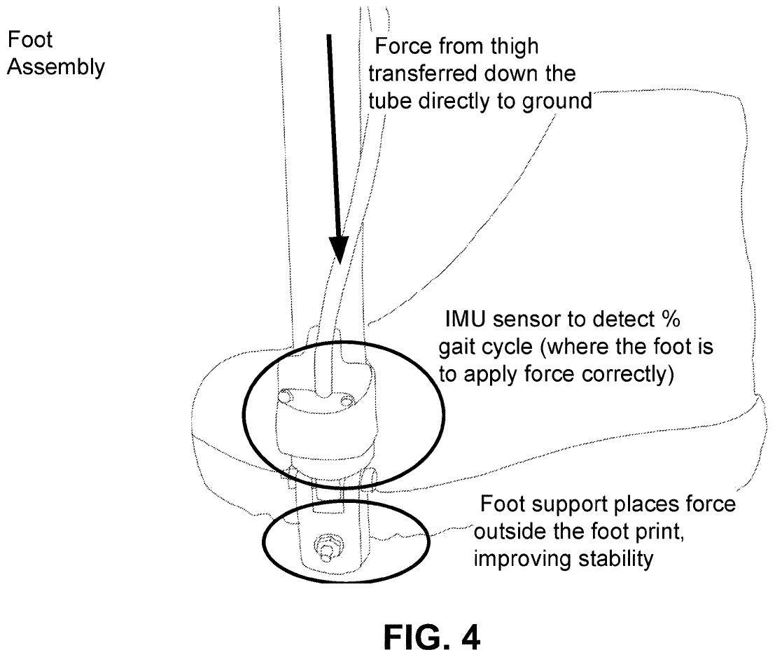

The motor pulls a tether to apply force and slide the carriage along the carbon pole (FIG. 3). The linear exoskeleton applies force to the leg by using the motor to wind a cord. This cord travels through a sheath, or a Bowden cable, to transmit forces directly to the linear exoskeleton. The Bowden cable sheath terminates on a carriage, which slides along the carbon pole of the linear exoskeleton. The cable runs over a pulley mounted on top of the exoskeleton pole, and terminates on the other side of the carriage with a load cell sensor that measures the force the device is applying to the exoskeleton. Thus, as force is applied by the motor, it attempts to wind in the cable on the exoskeleton, raising the carriage upwards toward the top of the carbon pole. The strap attached to the carriage transmits force to the person's thigh. The force through the thigh strap is transferred down the carbon pole directly to the ground, offloading the leg (FIG. 4). In one embodiment, the position of the load application relative to the foot can be altered.

The motor could be placed directly on the carriage or separately on another location of the body, such as in a fanny pack on the lower back. The previously described Bowden cable sheath would run between the motor and the carriage, regardless of the placement of the motor.

An Inertial Measurement Unit (IMU) is attached at the base of the pole to detect when the foot is on the ground and where the foot is within the gait cycle. The foot support places force outside the foot print, improving mediolateral stability of the person by widening their support beyond the foot outline. The placement of the foot support, where the linear exoskeleton comes in contact with the ground relative to the foot, could be actively changed to improve stability with a gantry system, slide, or other electro-mechanical apparatus.

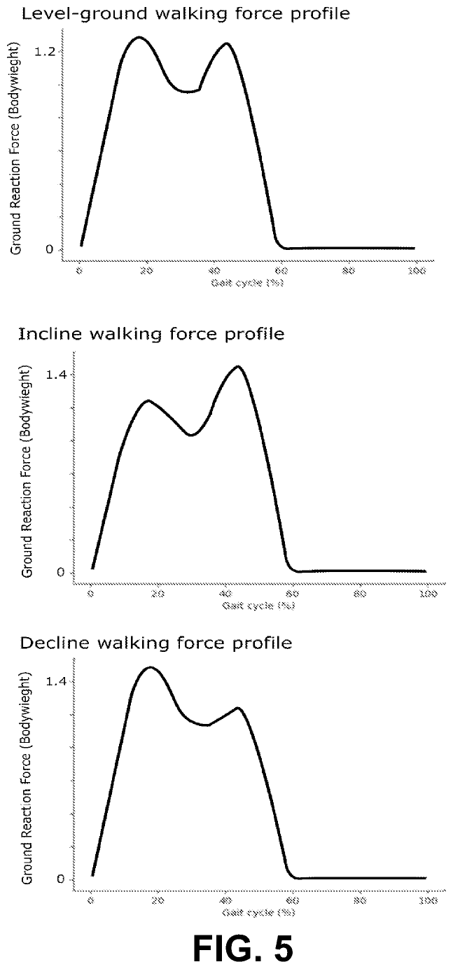

The linear exoskeleton applies forces to assist walking prescribed by a force profile applied by the leg during walking, and scaled to the desired assistance level (FIG. 5). When walking on an incline, the force profile has a lower first peak and higher second peak. When walking on a decline, the force profile has a higher first peak and a lower second peak. This is accomplished through sensors to know where a person is in their stance (starts when the heel strikes the ground and ends when the toe comes off). The sensors to track leg position are an IMU at the bottom of the carbon pole, but different types sensors could accomplish the same objective (e.g. a switch or a physical contact sensor in the shoe, a force sensor in the shoe, a pressure sensor in the shoe, an IMU on leg, EMG recording on leg, etc.).

To operate the linear exoskeleton, a person turns on the device, and stands still for the linear exoskeleton to perform a static calibration (reeling in cord slack in the device). A person then starts walking, wherein during the first steps (e.g. 6 steps) a dynamic calibration is performed to estimate the average time for each gait cycle. The idea is to take the average time of the past few steps to estimate the time one would think the current step will take. Based on the amount of time that has passed in the current step one could then compute what percent completed the step is (gait cycle percent) and then apply the corresponding force from the prescribed profile. Then during operation, the linear exoskeleton constantly calibrates based on the measured force from the load cell sensor on the carriage and the expected force profile.

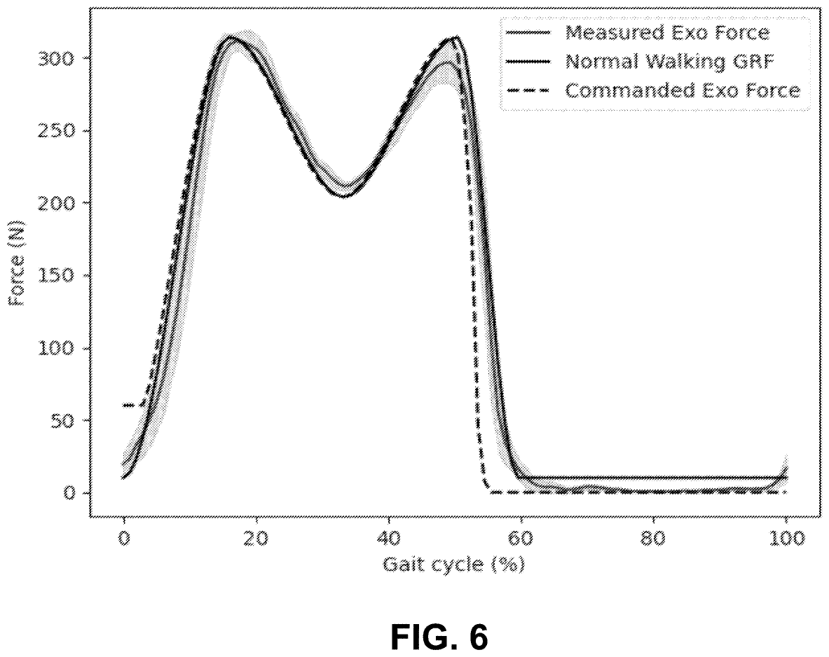

With the objective of the linear exoskeleton to assist the user by applying force in the direction of the leg, the assistance force profile is parameterized to be proportional to the natural ground reaction force between the foot and the ground during normal walking (or another type of movement such as running). The proportion of assistance is specified between 0% and 100%. An example of this is shown for a level-ground walking profile with 40% assistance of a person that weighs approximately 150 pounds (FIG. 6).

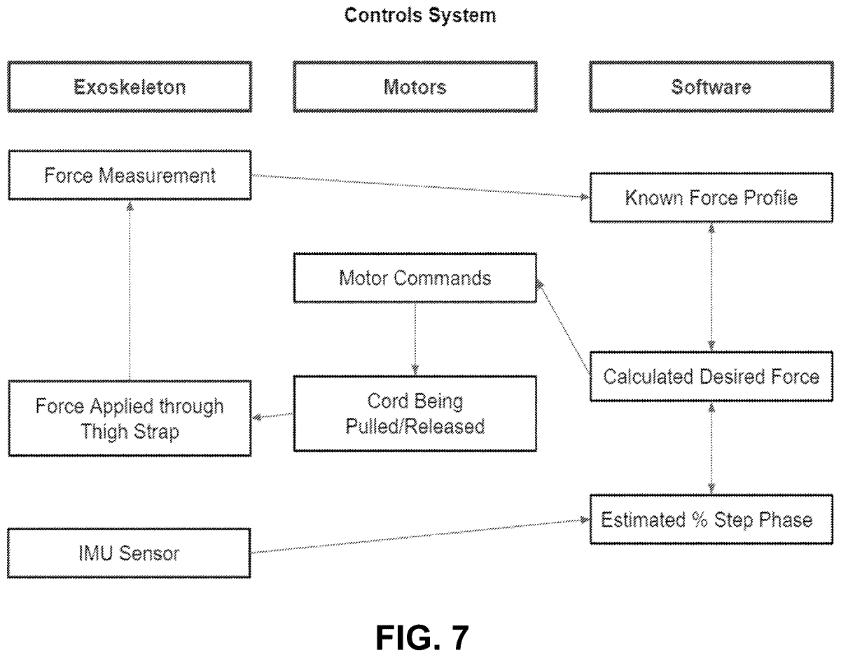

A linear exoskeleton controller applies this desired force profile by commanding a motor value to the motor and measuring the force applied to the exoskeleton using e.g. the load cell sensor on the exoskeleton, which is used as feedback to the controller (FIG. 7). An IMU sensor placed on the bottom of the pole is used to detect when the foot is in contact with the ground, and where the foot is during the step (referred to as the percent step phase, or percent stance phase). The IMU could measure acceleration, angular velocity, and magnetic field in 3D to provide this information.

### Results

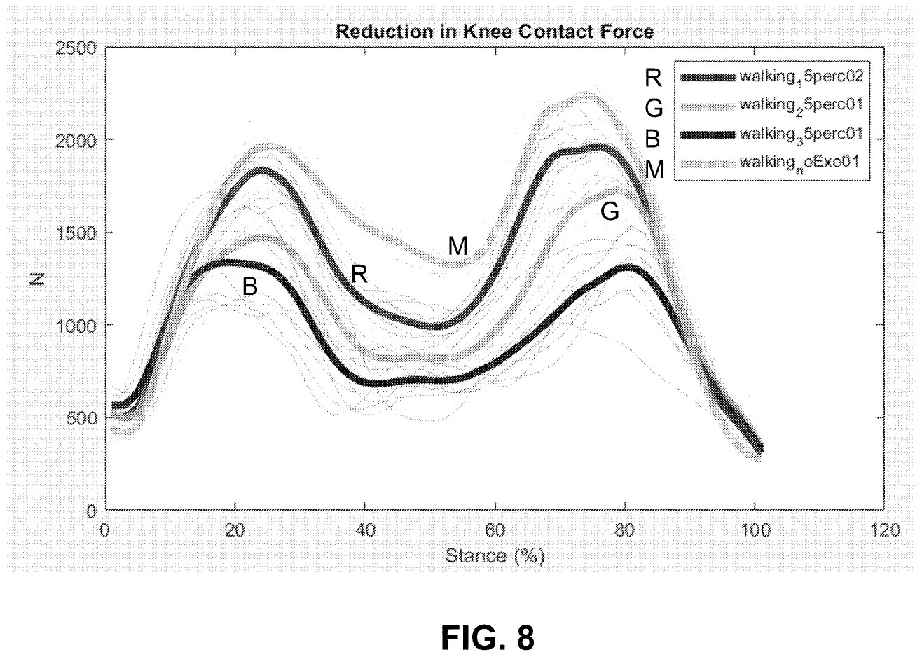

FIG. 8 shows result of an applied force which reduces the knee contact force approximately proportionally to how much assistance is applied by the user. For example, applying assistance of 30% of the person's body weight reduces the total knee contact force by approximately 30%.

What is claimed is:

1. A linear exoskeleton for reducing knee contact force, comprising:
(a) a pole with a distal end and a proximal end;
(b) a shoe attachment at the distal end of the pole and capable to be attached to a shoe;
(c) a carriage near the proximal end of the pole, wherein the pole is capable of moving relative to the carriage in up and down position;
(d) a thigh strap near the proximal end of the pole, wherein the thigh strap is capable to be affixed the pole to a person thigh and capable of positioning the pole adjacent to a person's leg;
(e) a mechanical assembly at the proximal end of the pole, wherein the mechanical assembly is mechanically connected to the carriage; and
(f) a motor controlling the relative distance between the assembly and the carriage, therewith capable of controlling an assistive force to the person and therewith capable of reducing a knee contact force of the person while the person is executing a leg movement and in contact with a ground surface.

2. The linear exoskeleton as set forth in claim 1, wherein the pole is a linear carbon pole.

3. The linear exoskeleton as set forth in claim 1, further comprising a sensor for sensing contact with the ground surface.

4. The linear exoskeleton as set forth in claim 1, further comprising a sensor for sensing the assistive force by the pole.

5. The linear exoskeleton as set forth in claim 1, further comprising a controller for controlling the motor according to a desired force contact profile of the leg movement and receiving feedback from one or more sensors regarding the assistive force and ground contact.

6. The linear exoskeleton as set forth in claim 5, wherein the controller further comprises a first calibration module for static calibration of the controller when no leg movement or ground contact is experienced.

7. The linear exoskeleton as set forth in claim 5, wherein the controller further comprises a second calibration module for dynamic calibration of the controller during execution of the leg movement.

8. The linear exoskeleton as set forth in claim 5, wherein the controller further comprises an input of body weight of the person and an algorithm to scale the desired force contact profile.

9. The linear exoskeleton as set forth in claim 5, wherein the controller further comprises a scaling factor and an algorithm to scale the desired force contact profile.

10. The linear exoskeleton as set forth in claim 5, wherein the controller further comprises input from one or more sensors and an algorithm to determine a type of leg movement, a desired force contact profile and a scaling factor to scale the desired force profile.

11. The linear exoskeleton as set forth in claim 1, wherein the attachment to the shoe is adjustable.

12. The linear exoskeleton as set forth in claim 1, wherein the positioning of the pole is at a lateral side of the person.
